(19) United States
(12) Patent Application Publication
(10) Pub. No.: US 2023/0077002 A1
(43) Pub. Date: Mar. 9, 2023

(54) Not a Candle, an Experience

(71) Applicant: Julia P. Walker, Debary, FL (US)

(72) Inventor: Julia P. Walker, Debary, FL (US)

(21) Appl. No.: 17/731,264

(22) Filed: Apr. 28, 2022

Related U.S. Application Data
(60) Provisional application No. 63/241,100, filed on Sep. 7, 2021.

(51) Int. Cl. A61N 5/06; A61L 9/03

(52) U.S. Cl. CPC A61N 5/0613; A61L 9/03; A61N 2005/0663

(57) ABSTRACT

The present invention discloses not a candle but an experience which provides far more benefits than traditional candles and electric candles and similar assemblies. The product is developed from eco-friendly and non-toxic materials and has crystallized glow which is 50% brighter than traditional lightning systems. The structure provides aromatherapy and light therapy and an also produce enough wavelength nanometers (480-520) to utilize this self-illuminating, highly scented, gel candle as a replacement for a light therapy box.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

The utility model relates to field of illumination technology and more particularly relates to a solar/non solar rechargeable aromatherapy and light therapy provided simultaneously via photoluminescence using a non-electric alternative light source.

### Description of the Related Art

Candles have been used to provide light for most of recorded history. Their historical importance is evidenced by the many terms that depend on candles for imagery: it is “better to light a single candle than curse the darkness”; “to burn a candle at both ends” means to waste; “to hold a candle” to someone is to assist him while he works; “not fit to hold a candle” to another means not equal to that person; “the game is not worth the candle” when it is not worth the cost of supplying candles for light. In literature, candles are sometimes used as metaphors for life itself: “out, out, brief candle, . . . ” Standard spermaceti candles were once used to measure units of illuminating power, giving rise to the terms “candela” and “candlepower.”

With the advent of other sources of artificial light, use of candles for lighting has lessened. Nowadays, candles are used primarily in religious services, memorial services, as decorative accessories, and to help create an atmosphere (for example, the proverbial dinner by candlelight).

There are many types of electronic candles and related advancements available on the market. They are usually simple luminaries with a candle shape and are simply used to provide lighting, such that they lack the aesthetic appearance, interest, taste and the easiness to create the atmosphere.

There are numerous inventions that can be seen providing a advancements in related field. For instance, a Solar candle light bearing Chinese patent 2,011,10492Y is issued to Chinese inventor. The utility model discloses a solar energy candle lamp, which comprises a candle-shaped shell, wherein a luminous component, a control circuit part and a charging battery are arranged in the candle-shaped shell, a solar cell panel is arranged on the bottom surface of the candle-shaped shell, the control circuit part comprises a sensing control switch, wherein the sensing control switch is communicated if the switch is positively arranged and is disconnected if the switch is reversely arranged. The candle lamp of the utility model has a lifelike candle outer shape and combustion effect, a solar energy charging device is utilized to provide power supply for the candle lamp, a special sensing control switch is arranged in the candle lamp, when the candle lamp is positively arranged, the control switch is closed and the candle lamp can be used, when the candle lamp is reversely arranged, the control switch is disconnected, the candle can only be charged, and the candle lamp is a product with convenient use, high safety, environmental protection and saved cost.

Another Chinese patent 2,019,92550U on Solar candle table light issued to Chinese inventor discloses a solar candle table light comprises a light holder, a light cover, a light wick, a light, a rechargeable battery and a control circuit board, wherein the light is electrically connected with the control circuit board, the light wick is arranged on the light cover and right faces towards the light, the light cover is provided with a solar panel, and the solar panel and the rechargeable battery are respectively electrically connected with the control circuit board. In daytime, sunshine can be absorbed by means of the solar panel to charge the rechargeable battery. At night, the light is powered by the rechargeable battery, and thereby using of batteries can be reduced, cost can be saved, and the environment pollution can be reduced.

Another Solar electronic candle bearing Slovenian patent SI2,287,8A2 aims to provide a solar electronic candle preferably a tombstone solar candle that is shaped like a wax candle in a plastic casing on the lid of the installed solar cells but allows for long-lasting operation. The solar electronic candle according to the invention has an electronic module that controls the operation of the candle and an appropriate accumulator battery as a source energy and on the cover of the housing installed solar cells, which during the daytime light charge the accumulator battery and at the same time represent the sensor to light the lights, for example, LEDs.

A Fragrance aromatherapy candle with twelve fragrances and lasting fragrance bearing Chinese patent 1,124,72859A is issued to East China University of Science and Technology. invention discloses a fragrance aromatherapy candle with twelve fragrance and lasting fragrance, which is prepared from the following components in parts by weight: mixing 95 parts of beeswax and 5 parts of dodecaperfume; the twelve perfume is prepared from the following components in percentage by mass: 5-10% of essence, 1-2% of a fragrance fixing agent, 0.5% of a flavoring agent and the balance of ethanol; the essence is twelve flower fragrances corresponding to the flower blossoming in May-Chang-Dou, and is selected from at least one of orchid, gardenia, litchi flower, sophora flower, peony, pomegranate flower, jasmine flower, lotus flower, jujube flower, sweet osmanthus flower, chrysanthemum flower and plum blossom; the fixative consists of isobutyric acid phenoxyethyl ester and benzyl alcohol in a mass ratio of (0.5-4) to 1; the flavoring agent is citrate polyether. The fragrance aromatherapy candle provided by the invention has the fragrance type corresponding to the specific flowery odour of twelve months, so that the specific pungent odor of the candle can be covered, and different enjoyment of the candle in different months in sense is endowed.

Another patent on electronic aromatherapy candle bearing WO patent 2,021,035688A1 is issued to French and Chinese inventors. The present invention relates to the technical field of lighting technologies, and disclosed is an electronic aromatherapy candle, comprising a base, a filling body having two ends formed with openings, an aromatherapy assembly, and a flame head. The aromatherapy assembly is arranged in an accommodating cavity of the base; specifically, the aromatherapy assembly comprises a solid perfume and a heating structure for heating the solid perfume; fragrance is uniformly diffused by means of heating of the heating structure; because the solid perfume is in a solid state, self-volatilization of the solid perfume can be effectively avoided, thereby saving resources; moreover, the base is detachably connected to the filling body so as to facilitate replacement of the solid perfume and maintenance of parts inside the aromatherapy candle; furthermore, a support plate is arranged in the filling body; the flame head is arranged in the filling body and extends above the support plate via a through hole; the flame head is configured to simulate a real flame burning scene; a paraffin layer is arranged on the support plate so as to simulate a paraffin surface scene close to the flame head during real flame burning, thereby improving the simulation degree of the aromatherapy candle.

An Environment-friendly aromatherapy candle and preparation method thereof bearing Chinese patent 1,111,17776A is issued to Suzhou Fengbei Biotech Co ltd. The invention discloses an environment-friendly aromatherapy candle, which is prepared from 100 parts of main combustion agent and 5-15 parts of aromatherapy microcapsule, wherein the main combustion agent is hydrogenated high-methyl ethyl carbonate, and the aromatherapy microcapsule is prepared from a core material, a wall material, an emulsifier and deionized water. The invention effectively utilizes the nontoxicity, high formability and sufficient combustion of the hydrogenated high methyl ethyl carbonate, thereby being capable of preparing the environment-friendly scented candle which is easy to have good plasticity, good formability and no pollution stimulation. The adopted microcapsule matrix can form particles with good dispersibility of the oil-in-water phase, and the loss of aroma components in the storage and transportation process is effectively reduced.

Another patent 1,025,1967Blon Aromatherapy candle waterlamp is issued to Pan Hua-Cheng Pan hua cheng. The patent relates to an aromatherapy candle waterlamp including: a waterlamp main body, a container, a bottom case and a top-cover. The main body is filled with a flowing liquid and set a hollow channel. The container is set in the top of the main body and the bottom case is fixedly connected with the bottom of the main body. A control circuit, a power module for supplying power, a light source, an electricity conductive wire, and an electric heating piece are set inside the bottom case. The light source irradiates toward the bottom of the main body. The electricity conductive wire is partially set through the hollow channel of the waterlamp main body and is electrically connected with an electric heating piece. The top-cover having a three-dimensional shape is set for correspondingly covering the top of the main body and is alternately set with a plurality of through holes.

It has to be noted that the current invention proposes an advancement in providing an upgradation in already present techniques. The current invention proposes a method to provide an aromatherapy and light therapy provided simultaneously via photoluminescence using a non-electric alternative light source. The assembly is charged through solar or indoor light and can maintain its effectiveness over 25 years.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of candle assembly and added value of conventional electronic and wax candle decorations with other functional changes.

It is another objective of the invention to provide an apparatus where Aromatherapy and light therapy is provided simultaneously via photoluminescence using a non-electric alternative light source.

It is also the object of the present invention is to provide an assembly which doesn't needs artificial source of light. It can be charged through solar or indoor lights.

A further object is to provide an assembly that is simple and easy to use and user doesn't need to work on it for several minutes until it starts functioning.

It is also the objective of invention to provide an advancement where eyes aren't harmed by looking at it directly.

It is further the objective of invention to provide “glow” which lasts from 6 hours to 12 hours, depending on the color. Furthermore, it can be recharged for up to 25 years.

A still further object is to provide an assembly which is aesthetically pleasing and can be used for multiple events.

It is a further object of the present invention to provide a durable and reliable candle effect which is waterproof and can be used as indoor/outdoor décor or night light.

Still yet another object of the present invention is to provide a new and improved assembly which provides in the apparatuses and methods of the prior art some of the advantages thereof, while simultaneously overcoming some of the disadvantages normally associated therewith.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

FIG. 1 depicts the candle assembly as per preferred embodiments of the invention.

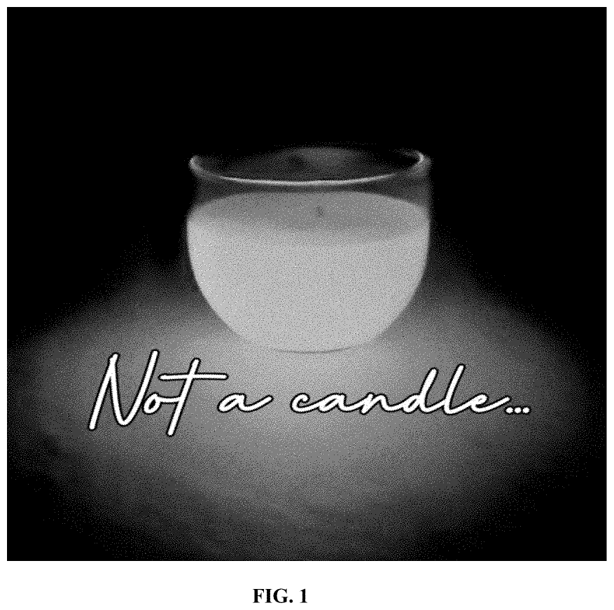

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

One aspect of the present application is directed to provide a system which proposes a candle with luminaries' effect designed in a candle shape and is used to provide lighting with aesthetic appearance, Aromatherapy and the easiness to create the atmosphere.

The history of the candle goes back to the middle ages when candles were made by hand. Most modern candles are machine fabricated by a molding process which forms a cylindrical mass of tallow or wax having embedded therein a twisted linen or cotton wick that burns to emit light.

To enhance the romantic appeal of candlelight, it is known to use scented candles which exude a pleasing fragrance as the candle burns. There are, however, a number of drawbacks with conventional scented candles in which the perfume oil is dispersed in the wax. There is a loss of perfume after prolonged storage; hence the scent, when the candle is burned, may be very faint. Moreover, the choice of scents is quite limited, for one needs for each distinctive scent a separate candle dedicated thereto.

Aromatherapy can relax your mind, relax and decompress. By massage, inhalation, hot compress, soaking, steaming and fumigation, aromatic essential oils (also called plant essential oils) are quickly integrated into the body's blood and lymph fluids, which can accelerate the body's metabolism and promote the regeneration of living cells Enhance the body's immunity, and then regulate the human nervous system, circulatory system, endocrine system, muscle tissue, digestive system and excretory system. Pure plant essential oil contains many polyphenols, which can stimulate the autonomic nerves in the body, make the endocrine system stable, natural and refreshing, and greatly increased vitality. Therefore, aromatherapy is more and more popular.

The present invention offers a more desirable and economic solution toward presentation of candles with enhanced features. The present invention comprises, in combination, a formula which is based on 16 ounces (one pound) of product, and can be halved, doubled, tripled, etc. By using a calibrated scale with tares for oz, lb, kg, and g, weigh one pound of Penreco Versagel:registered: CHP (high density) grade candle gel wax is designed for suspension of ingredients. and placed in a candle melting pot with the temperature set on ‘warm’.

Then temperature slowly increased to 232 degrees F. in small intervals, as to very slowly liquify the gel completely. It can take 30-45 minutes to complete the process. The temperature can be checked on regular intervals often with a candy thermometer attached to the inside opening of the melting pot.

While gel is melting, wick the desired size and quantity of containers you will need for 16 Oz of gel. For instance, if the size is a 5 oz container, you will need 3 containers and 3 wicks and stick-um tabs. You can use the last oz for 2 tealights that are 0.5 oz each.

The temp is checked again, however no stirring is required. Weigh out one ounce of a non-polarized fragrance oil with a minimum flash point of 170. Then weigh 6 grains of TechnoGlow ultra UV/glow powder. Set aside. When the thermometer reads 170, slowly add the fragrance oil and gently stir with a metal spoon for 4 minutes.

The Sprinkle powder around the top of melted gel and then “fold” it in the gel. This is actually suspended in the gel, as it does not mix or blend. Temperature should be at or near 232, it needs to be that temp to release bubbles. Once it reads 232, unplug immediately and DO NOTHING until it cools back down to 170. This is the perfect pour temp to ensure that the tops are smooth and glass-like. This also eliminates “pour bubbles and/or foam” The final product is cooled for 2 hours on a level surface, trim wick to ¼″. The final product is waterproof and can be used for indoor and outdoor. It has life up to 25 years and can be easily charged through solar or indoor lightning means.

The related drawings include a complete view of assembly. The drawing further shows the wicks points where wicks are rooted. With the increase in airflow from the bottom of the candle to the top, the scent of the burning candle is spread throughout the room more efficiently than a traditional candle.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1: An advance non electric lightning source;
a container having an open upper end and a bottom end;
means situated in said container for supporting a candle shaped assembly;
provision of Aromatherapy and light therapy via photoluminescence using a non-electric alternative light source;
a solar/indoor light chargeable assembly;
completely charged within 45 minutes;
available in six colors, ten aromatic scents and four sizes;

2: An advance assembly which can be used as a candle when lit, whereas it can also produce enough wavelength nanometers (480-520) to utilize this self-illuminating, highly scented, gel candle as a replacement for a light therapy box. This can be achieved unlit or lit.
